(19) United States
(12) Patent Application Publication
(10) Pub. No.: US 2022/0359054 A1
(43) Pub. Date: Nov. 10, 2022

(54) Calculation of Insulin Sensitivity Factors used to determine correction doses of insulin at varying levels of glycemia in human patients.

(71) Applicant: Christopher Richard Wilson, Oceanside, CA (US)

(72) Inventor: Christopher Richard Wilson, Oceanside, CA (US)

(21) Appl. No.: 17/736,383

(22) Filed: May 4, 2022

Related U.S. Application Data
(60) Provisional application No. 63/184,790, filed on May 6, 2021.

(51) Int. Cl. G16H 20/13; A61M 5/172; G16H 70/40; G16H 20/17

(52) U.S. Cl. CPC G16H 20/13; A61M 5/1723; G16H 70/40; G16H 20/17; A61M 2230/201

(57) ABSTRACT

This invention discloses a method of calculating insulin sensitivity factors for use in calculating insulin doses dynamically as a function of current blood glucose level based on a temporally-weighted average of the patient's total daily insulin dose, the pharmacodynamic and/or pharmacokinetic profile of the insulin used, and the patient's typical ratio of basal insulin to bolus insulin. When average total daily insulin and the other relevant variables are inserted into the equation, the result is a mathematical function that can be used to estimate the effects of each unit of insulin delivered on a patient's blood glucose level. This mathematical equation is valid over a wide range of diabetic conditions and could be widely used throughout the world at very little cost (and at considerable improvement) to the current technology in use for these purposes.

### CROSS REFERENCE TO RELATED APPLICATIONS:

This application claims priority from U.S. Provisional No. 63/184,790, tiled May 6, 2021, the contents of which are incorporated by reference.

### STATEMENT REGARDING FEDERALLY SPONSORED RESEARCH OR DEVELOPMENT:

This invention was not federally sponsored.

INVENTOR: Christopher Richard Wilson, citizen of Oceanside, Calif., and citizen of USA

ATTORNEY DOCKET: CWilson-U P-1

### BACKGROUND OF THE INVENTION

### Field of the Invention

This invention relates to a general field of medical medication dosing calculations, and more specifically, to a method by which insulin sensitivity factors, also known as correction factors, can be calculated to determine correction doses of insulin for patients with varying levels of glycernia.

### Brief Description of Invention

The invention is a formula and method of use of the formula to determine how insulin sensitivity factors are calculated so that they may be used to determine necessary doses of insulin to be given with the intent of returning a patient to the desired level of glycemia from a hyperglycemic state. The invention is relevant to a variety of human patients with varying levels of glycemia

### Statement of the Problem Solved

Diabetes is one of the most prevalent and serious disease states facing modern humans. It is a chronic health issue resulting from defects of glucose metabolism and the hormones that regulated, affecting how a person's body converts food into energy. As food is digested, most of it turns into glucose, a “sugar” and is released into a person's bloodstream. The hormone insulin acts as a “valve,” permitting glucose to enter cells for storage and use in metabolic processes. A person with diabetes either does not make sufficient insulin or does not use insulin efficiently, resulting in elevated concentrations of glucose in circulating blood. This can lead to serious health problems such as major adverse cardiovascular events, including heart attacks and strokes, nerve damage, kidney diseases, and vision impairment and loss.

It is estimated by the Centers for Disease Control and Prevention that at least 33% of adults in the United States have pre-diabetes (a condition of mildly elevated glycemia that does not meet the diagnostic threshold for diabetes), and over 10% have actual diabetes. It is estimated that nearly half a billion people in the world have diabetes, and this number is expected to double in the next 40 or 50 years. Additionally, diabetes has been identified as a post-acute complication of infection with the SARS-CoV-2 virus (COVID-19), which has been pandemic in the world since the year 2020. Thus, there is reason to expect that the rate of new diabetes diagnoses will accelerate in the tutu e. Thus, helping people with diabetes is extremely important.

The current state-of-the-art in the management and care of insulin-dependent diabetes—including Type 1 Diabetes (diabetes due to autoimmune-mediated pancreatic beta cell destruction), Type 2 Diabetes (diabetes due to insulin resistance) when complicated by insufficient pancreatic insulin secretion capacity, Type 3c Diabetes (diabetes due to pancreatic injury or partial or complete pancreatectomy), Cystic Fibrosis-Related Diabetes (CFRD), Idiopathic insulinopenic diabetes (diabetes of undetermined pathophysiology where the patient has insufficient pancreatic insulin secretion capacity), as well as some variants of MODY (Maturity-Onset Diabetes of the Young, also known as Monogenic Diabetes)—is a process known as automated insulin delivery. These systems operate by receiving data from a wearable glucose sensor which extrapolates blood glucose levels based on the levels of glucose in subcutaneous interstitial fluid, and a pump system which delivers a rapid-acting or ultra-rapid-acting insulin analog to the patient via a subcutaneous catheter. The software programs and methods which control insulin delivery in these systems—Interoperable Automated Glycemic Controllers (iAGCs) run on “black box” software, and are based on proprietary algorithms developed or licensed by individual insulin pump manufacturers, and/or by companies which market their controller software as a separate medical device.

However, insulin sensitivity (the expected reduction in blood glucose concentration resulting from the administration of a given dose of insulin) varies for individual patients in a dose-dependent manner, with each additional unit of insulin being less effective as a result of reduced insulin receptor expression in tissues responsive to insulin under conditions of exposure to increased circulating insulin concentrations (hyperinsulinemia).This reduction in the response to the larger doses of insulin required to correct hyperglycemia at elevated blood glucose levels results in insufficient doses of insulin being administered by automated insulin delivery systems under conditions of hyperglycemia.

This “black box” industry standard creates a serious problem in that the current “modern” technology is based upon an incorrect assumption that is relied on by the entire industry. For over 30 years, the configurable settings for insulin pumps have consisted of 3 user-configurable settings:

1. A “basal rate” of insulin that is (ideally) delivered continuously to support basic metabolic needs in the absence of food or other external factors

2. A “carb ratio” that defines for the pump the mass of carbohydrate eaten that is disposed of by each unit of insulin given in addition to the basal rate.

3. A “correction factor” or “insulin sensitivity factor” (ISF) which defines a static value by which each unit of insulin is assumed to reduce a patient's blood glucose level.

The incorrect assumption that all current insulin delivery systems make is that the insulin sensitivity factor, or ISF, is a static value. It is not. ISF declines logarithmically relative to the peak concentration of insulin achieved by a correction dose of insulin. This results in automated systems delivering either too much or too little insulin across various sections of the glycemic range. It also prevents current systems from correctly accounting for the effects of insulin that has already been administered to the patient in predicting and taking action to prevent hypoglycemia as a result of over-administration of insulin.

### Solution Presented by this Invention

The current invention that is described more fully in this patent application is a mathematical formula, and the user thereof, which corrects the current state-of-the-art erroneous calculations. Through identification, assessment of use of these calculations, the invention corrects those calculations allowing for more accurate dosing of insulins and more accurate predictions of the effects of insulin doses already administered to the patient.

Improvements Provided by this Invention: Description of how the Invention is an improvement over existing technology: Current insulin dosing calculations, whether done by humans or automatically by algorithms as part of automated insulin delivery systems, assume a static number for the “insulin sensitivity factor” (ISF), which is the reduction in blood glucose concentration estimated to result from the administration of each unit of insulin. This invention provides a mechanism for making that ISF vary relative to the desired reduction in in the patient's blood glucose concentration. Individual and business demographics that would use this Invention: Persons living with insulin-requiring diabetes. Physicians and other healthcare professionals who calculate insulin doses and ratios for patients to use in determining insulin dosing requirements for self-administered insulin doses. Companies designing, manufacturing, marketing, and distributing automated glycemic controller software as a component of automated insulin delivery systems. Companies and individuals designing “bolus calculator” software to assist patients in determining correct insulin dosing.

Description of the benefits of this invention to its users: More accurate insulin sensitivity estimations will allow for more accurate dosing calculations in automated insulin delivery systems and software products designed to assist patients and physicians in calculating doses. Additionally, patients calculating individual correction doses of insulin using this method are less likely to under- or over-dose their insulin, resulting in reduced glycemic variability, and increasing the amount of time spent in the optimal glycemic range.

Methods Used to Obtain Readings:

To obtain the various numbers required for use in this equation, a variety of techniques/machines are used. In case the reader in not familiar with various diabetes management technologies, readings used as inputs into this formula are obtained from either a “finger stick glucometer” which measures glucose concentration in whole blood via dynamic electrochemistry using either glucose oxidase or glucose dehydrogenase as a reagent or another equivalent method, or via a continuous glucose monitoring system (CGM), which provides estimates of blood glucose at regular intervals varying from one to five minutes.

There are currently multiple real-time CGM systems in the United States which have been approved by the Food and Drug Administration to have theft measurements used as a basis for dosing insulin (considered a “high-risk” m dication) without confirmatory finger stick glucose measurements: These include, but may not be limited to, the Dexcom G5, which was withdrawn from the market following the introduction of its successor system, the Dexcom G6, and the Eversense system from Senseonics. Additionally, the Freestyle Libre 2 system from Abbott is approved for use in dosing decisions, but it currently lacks the ability to natively transmit its readings to a connected automated insulin delivery system, instead relying on the patient to periodically “scan” the sensor with either a compatible smartphone or dedicated reader to obtain a history of the readings. It is anticipated that several additional glucose monitoring systems will be approved for use in the near-term future, including Dexcom's “G7” system, a new generation of Medtronics' “Guardian” CGM system, and the next generation of Abbott's Freestyle Libre system.

Thus, there has existed a long-felt need for a method by which more accurate insulin delivery estimations can be obtained for use in existing automated insulin delivery controller Nether integrated directly into an insulin pump or existing as a separate program on a controller device (including sr artphones), such that more accurate dosage calculations can be provided to patients with diabetes, and that this dosage calculation can change dynamically over time as the patient's insulin requirements and overall sensitivity to insulin vary. The result, for any system which calculates insulin dosages or ratios using the formula, would be more accurate and efficacious dosing of insulin to the patient, achieving superior glycemic control and corresponding improvements in patient health.

The current invention provides just such a solution by having a dynamic formula which takes into account previously obtained measurements, the pharmacodynamic profile of the specific type of rapid- or ultra-rapid-acting insulin analog being used, and other considerations such as the ratio of “basal,” or baseline insulin delivery, to “bolus,” or mealtime and correction insulin delivery, recent insulin delivery history, and other factors to produce a calculated insulin sensitivity value that can be incorporated into existing methods of dose calculation, including automated insulin delivery systems, software dose calculators, and other products to provide more physiologically accurate insulin dosing that is better matched to the needs of a patient with diabetes.

### Objects of the Invention

It is therefore an object of the present invention to provide a formula that produces an accurate insulin sensitivity estimate for each individual patient with diabetes.

Another object of the invention is to provide an insulin sensitivity estimate that dynamically changes in response to anticipated dose-dependent insulin resistance as the variables that are used in the equation change.

Additional objects of the invention include providing a formula which will result in more accurate dosing of insulin.

A further object of the invention is for use in monitoring systems for hospitalized patients which can, given knowledge of insulin doses administered by various routes, including subcutaneous and intravenous and their pharmacodynamic profiles to predict and proactively alert nurses and other healthcare professionals charged with patient care and monitoring preemptively, allowing them to prevent dangerous hypoglycemic conditions from occurring proactively.

Another object of the invention is to function as a component of a standalone system that monitors patient blood glucose levels continuously. The latest standalone CGM systems upload patient glycemic data to centralized computer systems run by the manufacturers of those systems for analysis and reporting. At least one of these systems uses that data to attempt to predict glycernic excursions (blood glucose values that are outside the desired “target range” of 70-180 mg/dL (3.8-10.0 mmol/L)) based on that historical data. This invention suggests a method that can be used to improve the accuracy of predictions made by these systems, as it relies not on historical trends, but on mathematical deductions and calculations based on the patient's current glycemic state, the specific type of insulin or insulins in use, and data about the doses of insulin that have been delivered to the patient over the preceding days and hours.

A final object of the invention is to provide a method by which existing glucose monitoring devices, glycemic controllers and insulin pumps can incorporate this formula into the software and thereby improve the accuracy of insulin dosing and predictions of future blood glucose levels.

### Statement of the Invention

Accordingly, one aspect of the invention includes a formula for accurately calculating insulin sensitivity in a human patient.

Preferably, the ISF (Insulin Sensitivity Factor or Correction Factor) is the number 1,800 divided by the product of a weighted average of the patient's total daily dose of insulin (TDD), an adjustment factor of a number between 0.1 and 1.9 representing the ratio of basal insulin to bolus insulin (BBR), and the natural logarithm (log base e) of 1 plus the patient's current blood glucose concentration measured in milligrams per dealer (BG) divided by a “Time to peak” factor (TTPF) that varies inversely with the time in minutes required for the particular insulin formulation in use to reach peak concentration in the patient's blood.

There has thus been outlined, rather broadly, the more important features of the invention in order that the detailed description thereof may be better understood, and in order that the present contribution to the art may be better appreciated. There are additional features of the invention that will be described hereinafter and which will form the subject matter of the claims appended hereto. The features listed herein and other features, aspects, and advantages of the present invention will become better understood with reference to the following description and appended claims. The accompanying drawings, which are incorporated in and constitute part of this specification, illustrate embodiments of the invention and, together with the description, serve to explain the principles of the invention.

It should be understood that while the preferred embodiments of the invention are described in some detail herein, the present disclosure is made by way of example only and that variations and changes thereto are possible without departing from the subject matter coming within the scope of the following claims, and a reasonable equivalency thereof, which claims I regard as my invention.

### BRIEF DESCRIPTION OF THE FIGURES

One preferred form of the invention will now be described with reference to the accompanying drawings.

FIG. 1 is a formula, according to the preferred form of the invention.

### DETAILED DESCRIPTION OF THE FIGURES

Many aspects of the invention can be better understood with references made to the drawings below. The components in the drawings are not necessarily drawn to scale. Instead, emphasis is placed upon clearly illustrating the components of the present invention. Moreover, like reference numerals designate corresponding parts through the several views in the drawings. Before explaining at least one embodiment of the invention, it is to be understood that the embodiments of the invention are not limited in their application to the details of construction and to the arrangement of the components set forth in the following description or illustrated in the drawings. The embodiments of the invention are capable of being practiced and carried out in various ways. In addition the phraseology and terminology employed herein are for the purpose of description and should not be regarded as limiting.

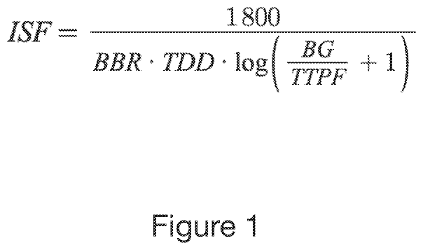

FIG. 1 is a mathematical formula calculating an insulin sensitivity factor to be used for determining correction doses of rapid-acting insulin, according to a preferred form of the invention. This initial determination is part of a treatment regimen for diabetic patients.

Definition Terms:

ISF=Insulin Sensitivity Factor (sometimes also called a Correction Factor)

BG —Glucose concentration in blood plasma (measured in milligrams per deciliter)

TDD=an average, which may be a weighted average, of the patient's total daily dose of insulin, expressed in units per day.

BBR=an adjustment factor, related to the ratio of basal insulin needs to bolus insulin needs for an individual patient.

TTPF=an adjustment factor which varies inversely with the time a particular formulation of insulin takes to reach peak concentration in a patient's bloodstream.

To use this method of calculating an insulin sensitivity factor to be used for determining correction doses of rapid-acting insulin, a temporally weighted average of the patient's total daily dose (TDD) of insulin is inserted into the denominator of the function at the spot labelled “TDD.” A basal/bolus ratio (BBR) adjustment factor related to the ratio of basal to bolus insulin typically required by the patient, and configurable by the patient or their healthcare provider is inserted into the denominator of the function at the spot labelled “BBR.” This adjustment factor will vary within the range of 0.1 to 1.9, with a patient whose total daily insulin usage is equally split between basal and bolus insulin having a “BBR” of approximately 1.0. A time-to-peak factor (TTPF) that varies inversely with the time in minutes it takes the particular insulin type and formulation used by the patient to reach its maximum concentration in the bloodstream is inserted at the spot labelled “TTPF.” The time-to-peak factor is estimated to be approximately 50 for standard insulin aspart preparations (Novologl Novorapid and equivalent generics), 55 for standard insulin lispro (Humalog, Admelog, equivalent generics), 65 for adjuvenated insulin aspart (Fiasp), and approximately 75 for adjuvenated insulin lispro (Lyumjev). A blood glucose concentration obtained from a glucometer or other approved method of determining blood glucose is then inserted into the function at the spot labelled BG. The calculation is then performed by dividing BG by TTPF, adding 1, taking the natural logarithm of the result, multiplying that intermediate result by the product of TDD and BBR to yield a final denominator for the equation and dividing 1800 by that final denominator to produce an estimated insulin sensitivity factor (ISF).

Unique Features of the Invention: Current dose calculations for insulin use a static number as an estimate of insulin sensitivity, independent of glycemia or the pharmacokinetic profile of the insulin in use. The method described provides a way to replace that static number used in dosage calculations with a mathematical function capable of computing insulin sensitivity dynamically across a wide glycemic range and range of rapid-acting and ultra-rapid-acting analog insulins, allowing for more accurate dosing of insulin in patients with insulin-requiring diabetes, resulting in significantly more accurate dosing during times where blood glucose is elevated (hyperglycemia).

In one embodiment of the invention, the invention provides a method of calculating an estimated sensitivity to a required dose of insulin to correct hyperglycemia, comprising: a formula as follows:

$$ISF = \frac{1800}{BBR \cdot TDD \cdot log ( \frac{BG}{TTPF} + 1 )}$$

where,
ISF=Insulin Sensitivity Factor (sometimes also referred to as a Correction Factor).
BBR=Basal/Bolus Ratio Factor, a number in the range of 0.1 to 1.9 which will vary based on the ratio of bolus insulin to basal insulin that the patient requires.
TDD=A temporally-weighted average of the patient's normal daily insulin use, expressed in units of insulin per day
BG=The patient's current blood glucose concentration (measured/expressed in milligrams per deciliter).
TTPF=Time-to-peak factor, a number that varies inversely with the time in minutes it takes for a particular insulin to reach peak concentration in the bloodstream

In this embodiment, a user of the invention can incorporate the formula into an item of existing software, where the item of existing software is used to determine insulin doses to be administered to the patient by an automated insulin delivery system comprised of a glucose monitor, an insulin pump, and an interoperable automated glycemic controller, whether integrated into the pump system or implemented as a separate device. Aternatively, the user can incorporate the formula into an item of new software, where the item of new software is used to control insulin delivery in an automated insulin delivery system where the item of new software determines a dosage of insulin to be administered to a patient. Finally, it is contemplated that a user could incorporate the formula into both a new software system and an existing software system, where both the new software system and the existing software system determine a dosage of insulin to be administered to the patient.

In this embodiment, the formula provides an accurate estimation of the sensitivity of the patient to a given insulin dose and the insulin sensitivity estimate is dynamic in nature, as one or more of the various components of the equation change. The method can be used in monitoring systems for hospitalized patients to better predict hypoglycemia or in a stand-alone system that monitors glucose levels continuously or intermittently. The method can be used in existing glucose monitors and insulin pumps to improve their predictions, performance, and dosing accuracy and contemplates a numerator where the numerator of the function has a range of 1,200 to 2,000, 2,000 to 2,800, or 1,600 to 2,000.

It is also contemlated that a user could take a second step of taking the ISF (Insulin Sensitivity Factor) and using it to compute a correct dosage of insulin.

It should be understood that while the preferred embodiments of the invention are described in some detail herein, the present disclosure is made by way of example only and that variations and changes thereto are possible without departing from the subject matter coming within the scope of the following claims, and a reasonable equivalency thereof, which claims I regard as my invention.

All of the material in this patent document is subject to copyright protection under the copyright laws of the United States and other countries. The copyright owner has no objection to the facsimile reproduction by anyone of the patent document or the patent disclosure, as it appears in official governmental records but, otherwise, all other copyright rights whatsoever are reserved.

That which is claimed:

1. A method of calculating an estimated sensitivity to a required dose of insulin to correct hyperglycemia, comprising:
a formula as follows:
$$ISF = \frac{1800}{BBR \cdot TDD \cdot log ( \frac{BG}{TTPF} )}$$
where,
ISF=Insulin Sensitivity Factor (sometimes also referred to as a Correction Factor).
BBR=Basal/Bolus Ratio Factor, a number in the range of 0.1 to 1.9 which will vary based on the ratio of bolus insulin to basal insulin that the patient requires.
TDD=A temporally-weighted average of the patient's normal daily insulin use, expressed in units of insulin per day
BG=The patient's current blood glucose concentration (measured/expressed in milligrams per deciliter).
TTPF=Time-to-peak factor, a number that varies inversely with the time in minutes it takes for a particular insulin to reach peak concentration in the bloodstream

2. The method of claim 1 where a user of the invention incorporates the formula into an item of existing software, where the item of existing software is used to determine insulin doses to be administered to the patient by an automated insulin delivery system comprised of a glucose monitor, an insulin pump, and an interoperable automated glycemic controller, whether integrated into the pump system or implemented as a separate device.

3. The method of claim 1, where a user of the invention incorporates the formula into an item of new software, where the item of new software is used to control insulin delivery in an automated insulin delivery system where the item of new software determines a dosage of insulin to be administered to a patient.

4. The method of claim 1, where a user of the invention incorporates the formula into both a new software system and an existing software system, where both the new software system and the existing software system determine a dosage of insulin to be administered to the patient.

5. The method of claim 1, where the formula provides an accurate estimation of the sensitivity of the patient to a given insulin dose.

6. The method of claim 5, where the insulin sensitivity estimate is dynamic in nature, as one or more of the various components of the equation change.

7. The method of claim 6, where the method can be used in monitoring systems for hospitalized patients to better predict hypoglycemia.

8. The method of claim 7, where the method can be used in a stand-alone system that monitors glucose levels continuously or intermittently.

9. The method of claim 7, where the method can be used in existing glucose monitors and insulin pumps to improve their predictions, performance, and dosing accuracy.

10. The method of claim 1, where the numerator of the function has a range of 1,200 to 2,000.

11. The method of claim 1, where the numerator of the function has a range of 2,000 to 2,800.

12. The method of claim 1, where the numerator of the function has a range of 1,600 to 2,000

13. The method of claim 1, additionally comprising a second step of taking the ISF (Insulin Sensitivity Factor) and using it to compute a correct dosage of insulin.

14. The method of claim 13, where a user of the invention incorporates the formula into an item of existing software, where the item of existing software is used to determine insulin doses to be administered to the patient by an automated insulin delivery system comprised of a glucose monitor, an insulin pump, and an interoperable automated glycemic controller, whether integrated into the pump system or implemented as a separate device.

15. The method of claim 13, where a user of the invention incorporates the formula into an item of new software, where the item of new software is used to control insulin delivery in an automated insulin delivery system where the item of new software determines a dosage of insulin to be administered to a patient.

16. The method of claim 13, where a user of the invention incorporates the formula into both a new software system and an existing software system, where both the new software system and the existing software system determine a dosage of insulin to be administered to the patient.

17. The method of claim 13, where the formula provides an accurate estimation of the sensitivity of the patient to a given insulin dose, where the insulin sensitivity estimate is dynamic in nature, as one or more of the various components of the equation change.

18. The method of claim 17, where the method can be used in monitoring systems for hospitalized patients to better predict hypoglycemia, where the method can be used in a stand-alone system that monitors glucose levels continuously or intermittently.

19. The method of claim 13, where the method can be used in existing glucose monitors and insulin pumps to improve their predictions, performance, and dosing accuracy.

20. The method of claim 13, where the numerator of the function has a range of 1,200 to 2,000.
